(19) United States
(12) Patent Application Publication
(10) Pub. No.: US 2013/0317329 A1
(43) Pub. Date: Nov. 28, 2013

(54) NON-INVASIVE, CONTINUOUS, REAL-TIME SYSTEM FOR DETERMINING TISSUE HYPOPERFUSION

(71) Applicants: Viachaslau Mikalayevich Barodka, Baltimore, MD (US); Daniel Berkowitz, Baltimore, MD (US)

(72) Inventors: Viachaslau Mikalayevich Barodka, Baltimore, MD (US); Daniel Berkowitz, Baltimore, MD (US)

(73) Assignee: The Johns Hopkins University, Baltimore, MD (US)

(21) Appl. No.: 13/982,664

(22) PCT Filed: Feb. 3, 2012
(86) PCT No.: PCT/US12/23852

§ 371 Date: Jul. 30, 2013

Related U.S. Application Data
(60) Provisional application No. 61/439,081, filed on Feb. 3, 2011.

(51) Int. Cl. A61B 5/1455; A61B 5/00

(52) U.S. Cl. CPC A61B 5/1455; A61B 5/742; A61B 5/746
USPC 600/322

(57) ABSTRACT

A non-invasive, continuous, real-time detection system for determining an amount of tissue hypoperfusion in a patient includes an illumination system adapted to illuminate a section of tissue of a patient with light comprising at least five wavelength components, a detection system arranged to detect light from the illumination system after the light has passed through the section of tissue, and a signal processing system adapted to communicate with the detection system. The signal processing system is configured to calculate a relative amount of each of five forms of hemoglobin compared to substantially total hemoglobin, the five forms of hemoglobin being oxy-hemoglobin, deoxy-hemoglobin, met-hemoglobin, carboxy-hemoglobin, and sulf-hemoglobin. The signal processing system outputs information concerning detected sulf-hemoglobin for determining the amount of tissue hypoperfusion, and the illumination system and the detection system are adapted to be at least one of stuck on, clamped on, or attached to an external region of the patient's body.

### CROSS-REFERENCE OF RELATED APPLICATION

This application claims priority to U.S. Provisional Application No. 61/439,081, filed Feb. 3, 2011, the entire contents of which are hereby incorporated by reference.

### BACKGROUND

1. Field of Invention

The field of the currently claimed embodiments of this invention relates to non-invasive, continuous, real-time detection systems for determining an amount of tissue hypoperfusion in a patient.

2. Discussion of Related Art

Spectroscopy is a common technique for measuring the concentration of organic and inorganic constituents of a solution. The theoretical basis of this technique is the Beer-Lambert law, which states that the concentration of an absorbent in solution can be determined by the intensity of light transmitted through the solution, knowing the path length, the intensity of the incident light, and the extinction coefficient at a particular wavelength. The minimum number of discrete wavelengths that are required to solve the equations are the number of significant absorbers that are present in the solution.

A practical application of this technique is pulse oximetry, which utilizes a noninvasive sensor to measure oxygen saturation (SpO2) and pulse rate. Pulse oximetry is a non-invasive method allowing one to monitor the oxygenation of a patient's hemoglobin. A sensor is placed on a thin part of the patient's body, usually a fingertip or earlobe, or in the case of an infant, across a foot. Light at red (660 nm) and infrared (940 nm) wavelengths is passed sequentially through the patient to a photodetector. The changing absorbance at each of the two wavelengths is measured, allowing determination of the absorbances due to the pulsing arterial blood alone, excluding venous blood, skin, bone, muscle, fat, and (in most cases) fingernail polish. Based upon the ratio of changing absorbance of the red and infrared light caused by the difference in color between oxygen-bound (bright red) and oxygen-unbound (dark red or blue, in severe cases) blood hemoglobin, a measure of oxygenation (the percentage of hemoglobin molecules bound with oxygen molecules) can be made.

In general, the sensor has light emitting diodes (LEDs) that transmit optical radiation of red and infrared wavelengths into a tissue site and a detector that responds to the intensity of the optical radiation after absorption (e.g. by transmission or transreflectance) by pulsatile arterial blood flowing within the tissue site. Based on this response, a processor determines measurements for SpO2, pulse rate, and can output representative plethysmographic waveforms. Thus, “pulse oximetry” as used herein encompasses its broad ordinary meaning known to one of skill in the art, which includes at least those noninvasive procedures for measuring parameters of circulating blood through spectroscopy. Moreover, “plethysmograph” as used herein, encompasses its broad ordinary meaning known to one of skill in the art, which includes at least data representative of a change in the absorption of particular wavelengths of light as a function of the changes in body tissue resulting from pulsing blood.

The same principle applies to a number of different wavelengths emitted. Any two wavelengths could be used to generate the NP wavelengths1/NP wavelength2 ratio. This is a generalization to multiple wavelengths of the ratio NP red/NPinfrared described above for two (red 660 nm and infrared 940 nm) wavelengths.

When a sensor is properly positioned on a tissue site, the detector only receives LED emitted light that has propagated through the tissue site after tissue scattering and absorption. Thus, a tissue profile should reflect the blood constituent absorption characteristics. At red and near IR wavelengths reduced hemoglobin (Hb) and oxygenated hemoglobin, HbO2 or “oxy-hemoglobin”, are the only significant absorbers normally present in the blood. Thus, typically only two wavelengths are needed to resolve the concentrations of Hb and Hb02, e.g. a red (RED) wavelength at 660 nm and an infrared (IR) wavelength at 940 nm. In particular, SpO2 is computed based upon a red ratio RedAC/RedDC and an IR ratio IRAC/IRDC, which are the AC detector response magnitude at a particular wavelength normalized by the DC detector response at that wavelength. The normalization by the DC detector response reduces measurement sensitivity to variations in tissue thickness, emitter intensity and detector sensitivity, for example. The AC detector response is a plethysmograph, as described above. Thus, the red and IR stands for “normalized plethysmograph”. In pulse oximetry, oxygen saturation is calculated from the ratio NPred/NPinfrared.

There are devices which use more complex method of additional LEDs for carboxhemoglobin measurements such as in U.S. Pat. Nos. 4,167,331; 5,355,880 and 5,412,100. Complex mathematical programs for calculating blood constituent levels from multiple variants are disclosed in U.S. Pat. Nos. 5,285,782 and 5,435,309. Both of these patents contemplate complete, multi-variant readings from their devices and the requisite microprocessor power to calculate the necessary algorithms.

Pulse oxymeters used to measure arterial oxygen saturation are available. U.S. Pat. No 4,653,498 (1987) describes a display monitor for use with a pulse oximeter of the type in which light of two different wavelengths is passed through body tissue, such as a finger, an ear or the scalp, so as to be modulated by the pulsatile component of arterial blood therein and thereby indicate oxygen saturation. Similarly, U.S. Pat. Nos. 4,621,643 and 4,700,798 and 4,770,179 describe disposable probes for use with pulse oximeters.

In U.S. Pat. No. 4,167,331 directed to a multi-wavelength incremental absorbance oximeter, light of two different wavelengths is passed through or reflected from a member of the body so as to be modulated by the pulsatile blood flow therein. The amplitudes of the alternating current components of the logarithms of the respective light modulations are compared by taking their molecular extinction coefficients into account so as to yield the degree of oxygen saturation. By adding a third wavelength of light, the percentage of other absorbers in the blood stream such as a dye or carboxyhemoglobin can be measured. Fixed absorbers reduce the amount of light that passes through or is reflected from the body member by a constant amount and so have no effect on the amplitudes of the alternating current components that are used in making the measurements.

However, there currently is no non-invasive, continuous, real-time detection system for determining an amount of tissue hypoperfusion in a patient. There thus remains a need for such systems.

### SUMMARY

A non-invasive, continuous, real-time detection system for determining an amount of tissue hypoperfusion in a patient according to an embodiment of the current invention includes an illumination system adapted to illuminate a section of tissue of a patient with light comprising at least five wavelength components, a detection system arranged to detect light from the illumination system after the light has passed through the section of tissue, and a signal processing system adapted to communicate with the detection system. The signal processing system is configured to calculate a relative amount of each of five forms of hemoglobin compared to substantially total hemoglobin, the five forms of hemoglobin being oxy-hemoglobin, deoxy-hemoglobin, met-hemoglobin, carboxy-hemoglobin, and sulf-hemoglobin. The signal processing system outputs information concerning detected sulf-hemoglobin for determining the amount of tissue hypoperfusion, and the illumination system and the detection system are adapted to be at least one of stuck on, clamped on, or attached to an external region of the patient's body.

A non-invasive, continuous, real-time method for determining an amount of tissue hypoperfusion in a patient includes illuminating a section of tissue of a patient with light comprising at least five wavelength components, detecting light from the illuminating after the light has passed through the section of tissue, calculating a relative amount of each of five forms of hemoglobin compared to substantially total hemoglobin with a signal processing system, the five forms of hemoglobin being oxy-hemoglobin, deoxy-hemoglobin, met-hemoglobin, carboxy-hemoglobin, and sulf-hemoglobin, and determining said amount of tissue hypoperfusion based on the relative amount detected sulf-hemoglobin.

### BRIEF DESCRIPTION OF THE DRAWINGS

Further objectives and advantages will become apparent from a consideration of the description, drawings, and examples.

FIG. 1 shows spectra of lysed human erythrocytes. Solid line: unmodified hemoglobin control (no H₂S, no cysteine, no cystalysin); dotted line: hemoglobin treated with 1.5 mM H₂S; dashed line: hemoglobin pretreated with cysteine and enzyme cystalysin which generates H₂S. (Kurzban, G. P., et al., Sulfhemoglobin formation in human erythrocytes by cystalysin, an L-cysteine desulfhydrase from Treponema denticola. Oral Microbiol Immunol, 1999. 14(3): p. 153-64)

FIG. 2 shows absorption spectra of deoxy-Hgb (______ ) oxy-Hgb ( ______ ______ ______), carboxy-Hgb (______.______), hgb (pH 7.0-7.4) (______ ______ ______ ) and sulf-Hgb (______ ______ ______), at room temperature. Absorptivity in mmol⁻¹ L cm⁻¹.

FIG. 3 is a schematic illustration of a non-invasive, continuous, real-time detection system for determining an amount of tissue hypoperfusion in a patient according to some embodiments of the current invention.

FIG. 4 is a schematic diagram illustrating some concepts of the current invention. At the left, multiple wavelengths of light are directed through tissue of the subject (center). The tissue contains blood. The detectors on the right detect non-absorbed portions of the light. The light can pass through in a transmission mode, or can be reflected and/or scatter back towards the direction of the emitters, depending on the particular embodiment.

FIG. 5 is a schematic illustration of a portion of a non-invasive, continuous, real-time detection system for determining an amount of tissue hypoperfusion in a patient according to an embodiment of the invention in which it is attached to a finger.

FIG. 6 is a schematic illustration of a portion of a non-invasive, continuous, real-time detection system for determining an amount of tissue hypoperfusion in a patient according to an embodiment of the invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated. The term “light” is intended to have a broad meaning to include both visible and non-visible regions of the electromagnetic spectrum. For example, infra-red light, near infra-red light, visible light and ultraviolet light are all intended to be included within the definition of the term light.

The reference to light that has passed through a section of tissue is intended to include situations in which light passes through the section of tissue in a transmission mode, i.e., in one side and out the other. For example, light can pass through a finger or ear lobe such that it enters through one surface and exits through an opposing surface without being reflected back towards the source of the light. In this case a detector is arranged at the opposing surface to detect the light that has passed through the section of tissue. It is also intended to cover situations in which the light is reflected and/or scattered back towards the source of the light. In this case, a separate or common component for detecting the light that has passed through the section of tissue is arranged at the initial surface close to or coincident with the source of light. In this case, the light travels through the section of tissue and is reflected or scattered back to pass through the section of tissue again. This can be considered a reflection mode of operation. For example, the placement of such a sensor system on a person's forehead would result in light being reflected and/or scattered back by the person's skull to be detected. The broad concepts of the current invention are not limited to these particular examples.

The endothelium is the innermost single cell layer of blood vessel. A healthy endothelium is the major source of a newly recognized vasoactive gas, hydrogen sulfide, which is required for normal vascular relaxation. Hypoxia leads to increased production of hydrogen sulfide. States of hypoperfusion such as septic and hemorrhagic shock lead to tissue hypoxia and results in elevated tissue levels of hydrogen sulfide. Hydrogen sulfide also mediates inflammatory responses partially via NFk-B stimulation. Interestingly, pharmacologic or genetic inhibition of hydrogen sulfide production has been shown to exert dramatic protection against sepsis, pancreatitis, acute lung injury, stroke and hemorrhagic shock. Increased H₂S production and bioavailability therefore may be tightly linked to tissue hypoperfusion and hypoxia. It is believed that H₂S produces a majority of it's effects via direct modifications of proteins via S-sulfhydration (S—SH bond formation on thiol groups).

Erythrocytes or red blood cells (RBCs) are in close contact with endothelium in the microvasculature. It has been shown that RBCs play an active role in H₂S production and bioavailability. Similarly H₂S produced in endothelial cells freely diffuses into RBCs. The RBC has been reported to be a buffer for other gaseous molecules NO and CO. The deoxygenated hemoglobin acts as a H₂S scavenger rapidly reacting with H₂S and forming sulf-Hgb. Thus there is a dynamic balance of SH-Hgb in RBCs depending on the levels of H₂5 production in endothelium and oxygenation states of Hgb depending on tissue metabolic rate. This represents a highly refined biological mechanism for local coupling of flow and metabolism.

Hgb has unique absorption spectra depending on whether molecules such as oxygen or carbon monoxide are bound to the heme group. This change in absorption is a basis of a methodology to non-invasively and continuously measure what percentage of Hgb is fully bound to oxygen. This technology is called oxymetry and is widely used in clinical practice for the measurement of oxygen saturation (an important component of integrated cardiovascular function). It has been claimed to be the most significant advancement in medical technology and resulted in dramatic increases in patient safety in anesthesia and medicine in general. It utilizes just two emission wavelengths around 600 nm and 900 nm to distinguish between oxygenated and deoxygenated Hgb. In order to detect simultaneously and separate the effect of carbon monoxide from oxygen, an additional wavelength has been added. That technology was labeled co-oxymetry. Similarly oxidizing Fe in Hgb from FeII into FeIII leads to met-hemoglobin formation with its unique absorption spectrum and ability to be detected by co-oxymetry using an additional wavelength specific for met-Hgb wavelengths.

When H₂S binds to Hgb, it's absortion spectrum changes in a systematic way, such that it is different from Hgb bound with O₂, CO, NO or from oxidized Fe in heme called met-hemoglobin. Hence, adding an additional wavelength to co-oxymetry at which absorption of SH-Hgb is different from other forms of Hgb will allow calculation of the amount of SH-Hgb. Higher levels of SH-Hgb will reflect higher systemic H₂S production levels and can serve as indicator of tissue hypoperfusion and severity of shock conditions such as, but not limited to, septic shock. Similarly, resolution of shock with improved tissue perfusion and oxygen delivery will result in less H₂S production and thus less formation of SH-Hgb. The co-oxymetry signal for SH-Hgb will therefore decrease.

A co-oxymetry technique according to an embodiment of the current invention can use a small probe placed on the skin of the finger or ear lobe, for example, similar to how oxymetry allows for continuous and non-invasive measurement of oxy-hemoglobin. Such a device can allow one to make inferences about adequacy of oxygen exchange in the lungs. Continuous and non-invasive measurement of SH-Hgb can allow one to make inferences of severity of shock and hypoperfusion states. Hemoglobin molecules are known for binding and transporting various gaseous molecules, such as oxygen, CO₂, CO and NO. Similarly, hemoglobin molecules are able to bind and transport H₂S (Silfa, E., et al., Orientation of the heme vinyl groups in the hydrogen sulfide-binding hemoglobin I from Lucina pectinata. Biospectroscopy, 1998. 4 (5): p. 311-26; Pietri, R., et al., Factors controlling the reactivity of hydrogen sulfide with hemeproteins. Biochemistry, 2009. 48 (22): p. 4881-94). Hemoglobin has been shown to be sulfhydrated by H₂S to form sulf-Hgb by highly reactive Fe2+ in the heme portion and by binding of H₂S to disulfide or free thiols on the globin portion (Arp, A. J. and J. J. Childress, Sulfide Binding by the Blood of the Hydrothermal Vent Tube Worm Riftia pachyptila. Science, 1983. 219 (4582): p. 295-7; Wang, R., Two's company, three's a crowd: can H2S be the third endogenous gaseous transmitter? FASEB J, 2002. 16 (13): p. 1792-8; Zal, F., et al., S-Sulfohemoglobin and disulfide exchange: the mechanisms of sulfide binding by Riftia pachyptila hemoglobins. Proc Natl Acad Sci USA, 1998. 95 (15): p. 8997-9002). Whether sulfhydrated heme is able to trans-sulfhydrate the free thiol in the globin portion similar to NO is unknown. The physiologic role of sulfhydrated Hgb is unknown. It is likely that H₂S is eventually released from the RBC into systemic circulation to regulate vascular function. Since the RBC H₂S pool is in equilibrium with the endothelial cell H₂S pool, detection of SH-Hgb can serve as a surrogate for the H₂S production by the endothelium according to an embodiment of the current invention.

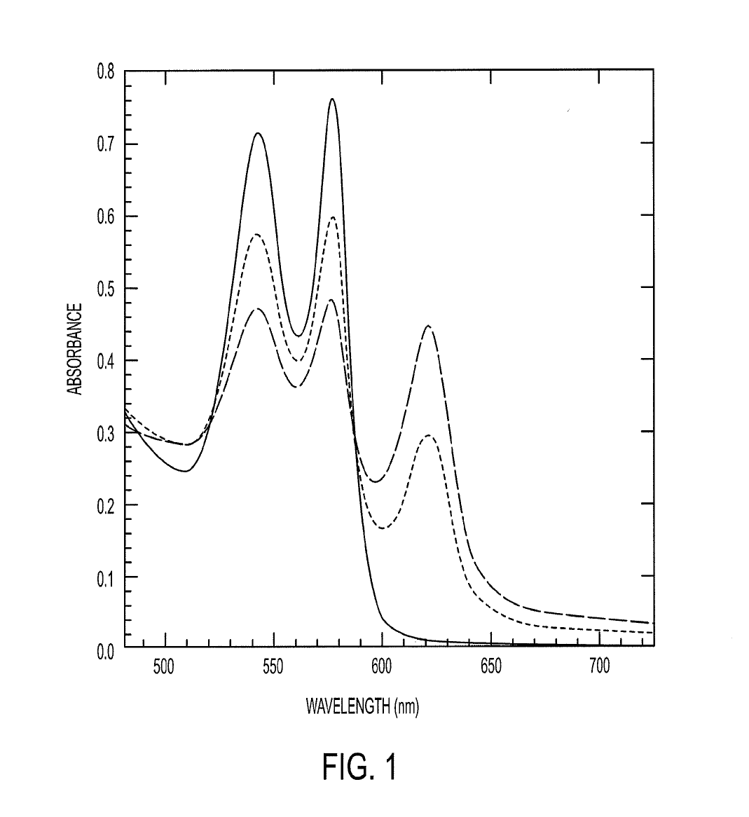

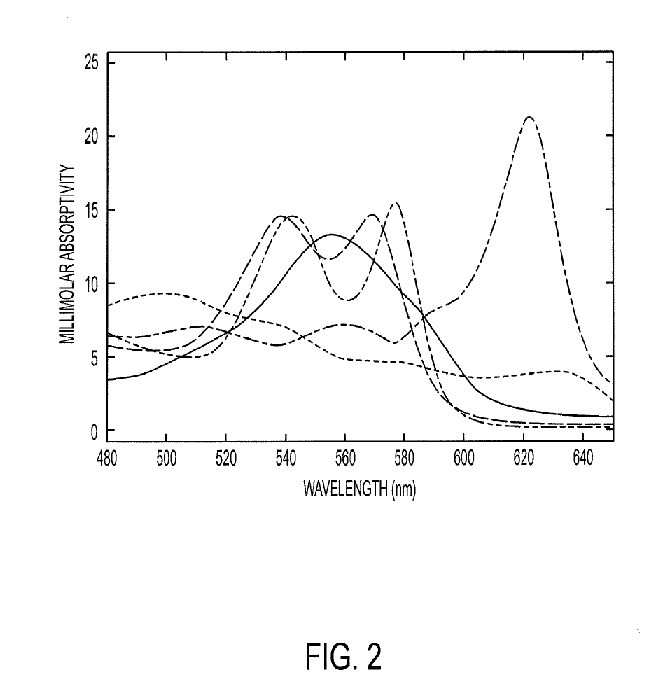

The absorption spectrum of sulf-hemoglobin is different from that of oxy-Hgb (see FIGS. 1 and 2) which can allow discrimination of the two forms of Hgb by co-oxymetry. Importantly, the maximum difference in the absorption spectrum of Hgb compared to sulf-hemoglobin is at 621 nm. This is a different wavelength from the wavelength at which the maximum difference of Oxy-Hgb vs deoxyHgb is observed (600 nm and 900 nm) (Kurzban, G. P., et al., Sulfhemoglobin formation in human erythrocytes by cystalysin, an L-cysteine desulfhydrase from Treponema denticola. Oral Microbiol Immunol, 1999. 14 (3): p. 153-64). This can permit precise measurements of sulf-hemoglobin according to some embodiments of the current invention. The site for H₂S modification of Hgb is believed to be located in the periphery of the heme moiety, rather than a direct reaction with the heme iron (Chatfield, M. J. and G. N. La Mar, 1H nuclear magnetic resonance study of the prosthetic group in sulfhemoglobin. Arch Biochem Biophys, 1992. 295 (2): p. 289-96). As a result, sulf-hemoglobin in contrast to met-hemoglobin is able to bind oxygen. Oxysulfhemoglobin and deoxysulfhemoglobin have a slightly different absorption peaks being at 623 nm and at 619 nm respectively (Carrico, R. J., J. Peisach, and J. O. Alben, The preparation and some physical properties of sulfhemoglobin. J Biol Chem, 1978. 253 (7): p. 2386-91; Carrico, R. J., W. E. Blumberg, and J. Peisach, The reversible binding of oxygen to sulfhemoglobin. J Biol Chem, 1978. 253 (20): p. 7212-5). Importantly, oxygen binding to sulf-hemoglobin is weaker compare to unmodified hemoglobin, suggesting allosteric regulation of oxygen transport by H2S and favoring unloading of oxygen to peripheral tissues (Tomoda, A., A. Kakizuka, and Y. Yoneyama, Oxidative and reductive reactions of sulphhaemoglobin with various reagents correlated with changes in quaternary structure of the protein. Biochem J, 1984. 221 (3): p. 587-91). The heme iron in sulf-hemoglobin can undergo oxidation similar to oxyhemoglobin, producing sulfinethemoglobin with its unique absorption spectrum (a new small peak at 717 nm) (Carrico, R. J., J. Peisach, and J. O. Alben, The preparation and some physical properties of sulfhemoglobin. J Biol Chem, 1978. 253 (7): p. 2386-91). Exposure of Hgb to H₂S results mostly in production of sulfhemoglobin and not in sulfmethemoblobin. The use of multi-wavelength analysis of Hgb from human samples for clinical quantification of sulfhemoblobin and distinguishing it from oxyhemoglobin, methhemoblobin, CO-Hgb has been successfully performed (Zwart, A., E. J. van Kampen, and W. G. Zijlstra, Results of routine determination of clinically significant hemoglobin derivatives by multicomponent analysis. Clin Chem, 1986. 32 (6): p. 972-8). It was reported that a normal human level of sulf-hemoglobin is below 0.4%, which is consistent with low production of H₂S under normal perfusion states. However, the use of sulf-hemglobin levels in the blood in order to determine levels of hypoperfusion were not recognized. Furthermore, the above-noted data were taken in a laboratory setting and not using a non-invasive, continuous, real-time detection system.

Red blood cells (RBCs) act as sink for highly reactive gaseous molecules such as NO, CO, and H₂S and hence regulate the availability of hydrogen sulfide. The H₂S diffused into RBCs might be partitioning between H₂S bound to free thiol and to the disulfides of the hemoglobin or glutathione.

The significantly increased production of H₂S during shock conditions plays a crucial and unfortunately detrimental role. In contrast, increased levels of H₂S have been shown to be protective and reduced myocardial infarction. However, there are no methods to measure H₂S bioavailability and there is still on-going debate in the literature as to what is the physiologically relevant concentration of H₂S in blood. Novel drugs are under development both to augment and inhibit H₂S production. Therefore, an embodiment of the current invention provides a novel non-invasive, continuous and real-time co-oxymetry technique to measure H₂S bioavailability utilizing the SH-Hgb absorption spectrum. Levels of SH-Hgb can thus provide both prognostic and diagnostic information in variety of critical diseases such as, sepsis, hemorrhagic shock, pancreatitis, acute lung injury, stroke and myocardial infarction.

H₂S is a freely diffusible molecule. Increased amounts of H₂S produced during periods of hypoperfusion will diffuse not only into the RBCs but also into the muscle, where myoglobin, another heme containing protein, is located. Myoglobin, similar to hemoglobin, binds with gaseous molecules e.g. (O₂ and CO) and it has been shown to be able to bind H₂S, producing sulf-myoglobin. As in hemoglobin, binding of gaseous molecules to myoglobin changes it's absorption spectrum. Hence measurements of specific absorption spectrum of sulf-myoglobin in the muscles can provide another technique to assess the H₂S bioavailability and to make inferences on the tissue perfusion.

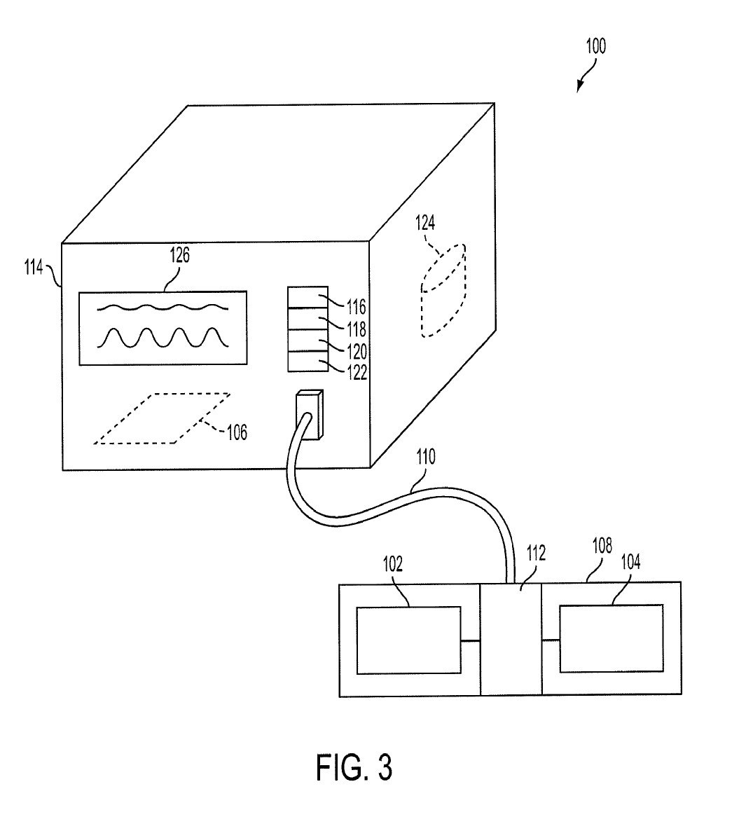

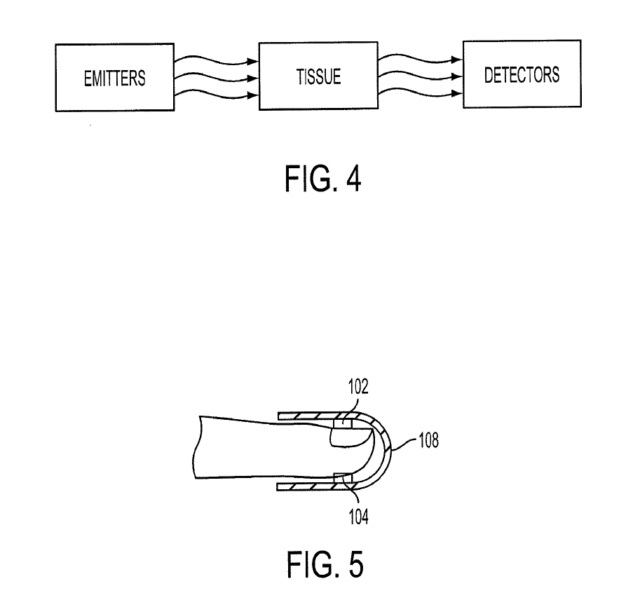

FIG. 3 is a schematic illustration of a non-invasive, continuous, real-time detection system 100 for determining an amount of tissue hypoperfusion in a patient according to an embodiment of the current invention. The non-invasive, continuous, real-time detection system 100 includes an illumination system 102 adapted to illuminate a section of tissue of a patient with light comprising at least five wavelength components. The non-invasive, continuous, real-time detection system 100 also includes a detection system 104 arranged to detect light from the illumination system 102 after the light has passed through the section of tissue of the patient. The non-invasive, continuous, real-time detection system 100 also includes a signal processing system 106 adapted to communicate with the detection system 104. The signal processing system 106 is configured to calculate a relative amount of each of five forms of hemoglobin compared to substantially total hemoglobin. The five forms of hemoglobin are oxy-hemoglobin, deoxy-hemoglobin, met-hemoglobin, carboxy-hemoglobin, and sulf-hemoglobin. (See also, FIG. 4.) The signal processing system 106 is also configured to output information concerning detected sulf-hemoglobin for determining the amount of tissue hypoperfusion. The illumination system 102 and the detection system 104 are adapted to be at least one of stuck on, clamped on, or attached to an external region of the patient's body.

There are four major modifications of hemoglobin, which along with the unmodified form, are as follows:
1) heme portion has reduced Fe2+ and it is not bound to anything. It is called deoxy-Hgb
2) heme portion has reduced Fe2+ and it bound to oxygen. It is called oxy-Hgb
3) heme potrion has oxydized Fe3+. It is called met-Hgb (it cannot bind to anything)
4) heme portion has reduced Fe2+ and it is bound to carbon monoxide. It is called carboxy-Hgb.
5) heme portion has reduced Fe2+ and it is bound to hydrogen sulfide. It is called sulf-Hgb.

Together they constitute 99.99% of all Hgb (e.g., for venous blood of a healthy adult 75% oxy-Hgb, 23% deoxy-Hgb, 2% met-Hgb, 0.5% carboxy-Hgb, 0.04% Sulf-Hgb). Therefore, measured values of these five forms of hemoglobin can be used as an approximation to the total amount of hemoglobin. This is intended to be within the definition of the term “substantially total hemoglobin”. Substantially total hemoglobin is intended to cover any suitable approximation to the total amount of hemoglobin for a particular application. It can be based on measured or other values.

Since the sulf-Hgb has the lowest concentration out of the usually encountered forms of hemoglobin, the non-invasive, continuous, real-time detection system 100 measures all five Hgb modifications to accurately calculate the sulf-Hgb concentration. Hence, at least five different wavelengths are used. For example, each wavelength can be selected at the peak absorption for each type of Hgb (e.g., sulf-Hgb has peak absorption at 621 nm). In some embodiments, it may be sufficient to be within 10 nm of the peak, or within 5 nm of the peak, or within 1 nm of the peak. Based on absorption at each wavelength, the signal processing system 106 calculates the percentage of each Hgb fraction relative to the total Hgb.

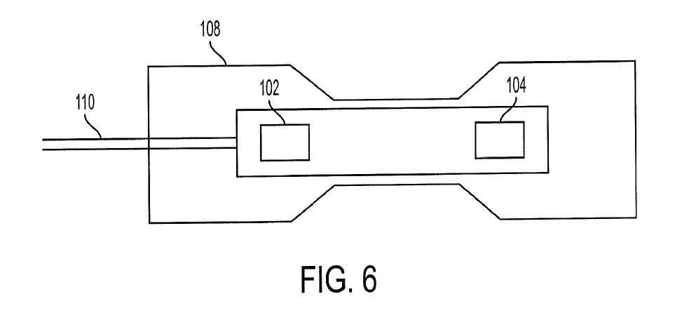

The illumination system 102 can include one or more LEDs, for example, to emit light in at least five distinct wavelength bands of light. Alternatively, solid state lasers could be used in place of LEDs; however, LEDs are sufficient for many applications. The detection system 104 can also include semiconducting components, such as, but not limited to photodiodes. The illumination system 102 and the detection system 104 can be incorporated into a variety of structures, such as structure 108 to be clamped onto a finger, toe or ear, for example, similar to a clothes pin or other spring activated structure. However, the concepts of the current invention are not limited to this particular example. Other embodiments of structure 108 can include flexible structures that have adhesive, for example (FIGS. 5 and 6). In other embodiments, the illumination system 102 and detection system 104 can be held in place with a separate component such as a tie, elastic band, etc. Further embodiments can incorporate the illumination system 102 and detection system 104 into articles that can be worn such as head-bands, helmets, hats, gloves, etc. The broad concepts of the current invention are not limited to these particular examples.

In some embodiments, the illumination system 102 and the detection system 104 can be connected to the signal processing system 106 by an electrical cable 110 through interconnect 112, for example. Alternatively, the illumination system 102 and the detection system 104 can be connected to the signal processing system 106 by a wireless connection. Further electronic components can also be connected to the illumination system 102 and/or the detection system 104 such as, but not limited to, a sensor controller.

In some embodiments, the non-invasive, continuous, real-time detection system 100 can also include a display system 114 configured to communicate with the signal processor 106 to display the information concerning detected sulf-hemoglobin for determining the amount of tissue hypoperfusion. For example, the display system 114 can display the relative amount of sulf-hemoglobin 116 detected. In some embodiments, the display system 114 can also display one or more of the relative amount of oxy-hemoglobin 118, met-hemoglobin 120 or carboxy-hemoglobin 122 detected.

In some embodiments, the signal processor 106 can be further configured to compare the relative amount of sulf-hemoglobin to a threshold value and output an alarm signal to the display system when the threshold is exceeded. The alarm can be visible, audible, a vibration or any other suitable way of signal an alarm.

In some embodiments, the non-invasive, continuous, real-time detection system 100 can also include a data storage unit 124 configured to communicate with the signal processor 106. The signal processor 106 can be configured to store a plurality of calculated relative amounts of sulf-hemoglobin over a period of time to form a trend of relative amounts of sulf-hemoglobin as a function of time, and the display system 114 can be configured to display 126 the trend of relative amounts of sulf-hemoglobin over the period of time.

In some embodiments, the signal processor 106 can be further configured to store a plurality of calculated relative amounts of met-hemoglobin over a period of time to form a trend of relative amounts of met-hemoglobin as a function of time and to compare the trend of relative amounts of sulf-hemoglobin to the trend of relative amounts of met-hemoglobin to distinguish between hypoperfusion due to sepsis shock and hypoperfusion due to cardiogenic shock. In the case of sepsis shock, the sulf-hemoglobin relative amount and the met-hemoglobin relative amount will both increase at the same time. In the case of cardiogenic shock, the relative amount of met-hemoglobin will remain relatively unchanged as the sulf-hemoglobin increases. These trends in combination with thresholding can provide predictors of not only hypoperfusion, but also potential causes.

The embodiments discussed in this specification are intended to explain concepts of the invention. However, the invention is not intended to be limited to the specific terminology selected and the particular examples described. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A non-invasive, continuous, real-time detection system for determining an amount of tissue hypoperfusion in a patient, comprising:
an illumination system adapted to illuminate a section of tissue of a patient with light comprising at least five wavelength components;
a detection system arranged to detect light from said illumination system after said light has passed through said section of tissue; and
a signal processing system adapted to communicate with said detection system,
wherein said signal processing system is configured to calculate a relative amount of each of five forms of hemoglobin compared to substantially total hemoglobin, said five forms of hemoglobin being oxy-hemoglobin, deoxy-hemoglobin, met-hemoglobin, carboxy-hemoglobin, and sulf-hemoglobin,
wherein said signal processing system outputs information concerning detected sulf-hemoglobin for determining said amount of tissue hypoperfusion, and
wherein said illumination system and said detection system are adapted to be at least one of stuck on, clamped on, or attached to an external region of said patient's body.

2. A non-invasive, continuous, real-time detection system for determining an amount of tissue hypoperfusion in a patient according to claim 1, wherein one of said five wavelength components is about 621 nm.

3. A non-invasive, continuous, real-time detection system for determining an amount of tissue hypoperfusion in a patient according to claim 1, wherein each of said five wavelength components corresponds to substantially a peak absorption wavelength of an absorption curve for a corresponding one of each of said five forms of hemoglobin.

4. A non-invasive, continuous, real-time detection system for determining an amount of tissue hypoperfusion in a patient according to claim 3, wherein one of said five wavelength components is about 621 nm.

5. A non-invasive, continuous, real-time detection system for determining an amount of tissue hypoperfusion in a patient according to claim 1, wherein said substantially total hemoglobin is determined to be a sum of amounts of all of said five forms of hemoglobin detected.

6. A non-invasive, continuous, real-time detection system for determining an amount of tissue hypoperfusion in a patient according to claim 1, further comprising a display system configured to communicate with said signal processor to display said information concerning detected sulf-hemoglobin for determining said amount of tissue hypoperfusion.

7. A non-invasive, continuous, real-time detection system for determining an amount of tissue hypoperfusion in a patient according to claim 6, wherein said signal processor is configured to output said relative amount of sulf-hemoglobin to be displayed by said display system.

8. A non-invasive, continuous, real-time detection system for determining an amount of tissue hypoperfusion in a patient according to claim 7, wherein said signal processor is further configured to compare said relative amount of sulf-hemoglobin to a threshold and to output an alarm signal to said display system when said threshold is exceeded.

9. A non-invasive, continuous, real-time detection system for determining an amount of tissue hypoperfusion in a patient according to claim 8, wherein said display system displays at least one a visible or an audible alarm in response to said alarm signal output by said signal processor when said when said threshold is exceeded.

10. A non-invasive, continuous, real-time detection system for determining an amount of tissue hypoperfusion in a patient according to claim 6, further comprising a data storage unit configured to communicate with said signal processor, wherein said signal processor is configured to store a plurality of calculated relative amounts of sulf-hemoglobin over a period of time to form a trend of relative amounts of sulf-hemoglobin as a function of time, and
wherein said display system is configured to display said trend of relative amounts of sulf-hemoglobin over said period of time.

11. A non-invasive, continuous, real-time detection system for determining an amount of tissue hypoperfusion in a patient according to claim 10, wherein said signal processor is configured to store a plurality of calculated relative amounts of met-hemoglobin over a period of time to form a trend of relative amounts of met-hemoglobin as a function of time, and
wherein said signal processor is further configured to compare said trend of relative amounts of sulf-hemoglobin to said trend of relative amounts of met-hemoglobin to distinguish between hypoperfusion due to sepsis shock and hypoperfusion due to cardiogenic shock.

12. A non-invasive, continuous, real-time method for determining an amount of tissue hypoperfusion in a patient, comprising:
illuminating a section of tissue of a patient with light comprising at least five wavelength components;
detecting light from said illuminating after said light has passed through said section of tissue;
calculating a relative amount of each of five forms of hemoglobin compared to substantially total hemoglobin with a signal processing system, said five forms of hemoglobin being oxy-hemoglobin, deoxy-hemoglobin, met-hemoglobin, carboxy-hemoglobin, and sulf-hemoglobin; and
determining said amount of tissue hypoperfusion based on said relative amount detected sulf-hemoglobin.

13. A non-invasive, continuous, real-time method for determining an amount of tissue hypoperfusion in a patient according to claim 12, further comprising comparing said relative amount of sulf-hemoglobin to a threshold and signaling an alarm when said threshold is exceeded.

14. A non-invasive, continuous, real-time method for determining an amount of tissue hypoperfusion in a patient according to claim 12, further comprising comparing a trend of relative amounts of sulf-hemoglobin to a trend of relative amounts of met-hemoglobin over time both detect hypoperfusion and to distinguish between hypoperfusion due to sepsis shock and hypoperfusion due to cardiogenic shock.
